(19) United States
(12) Patent Application Publication
(10) Pub. No.: US 2008/0253968 A1
(43) Pub. Date: Oct. 16, 2008

(54) IN VIVO EXAMINATION METHOD

(75) Inventors: Masahiro Oba, Hino-shi (JP); Nobuhiko Onda, Hachioji-shi (JP)

Correspondence Address: PILLSBURY WINTHROP SHAW PITTMAN, LLP, P.O. BOX 10500, MCLEAN, VA, 22102, US

(73) Assignee: OLYMPUS CORPORATION, Tokyo (JP)

(21) Appl. No.: 12/100,078

(22) Filed: Apr. 9, 2008

(30) Foreign Application Priority Data
Apr. 13, 2007 (JP) 2007-105936

(51) Int. Cl. A61K 49/00

(52) U.S. Cl. 424/9.6

(57) ABSTRACT

It is possible to perform quantitative examination of biological tissue even with a solution composed of a near-infrared fluorescent dye and a polypeptide. An image acquired before administering a reagent is subtracted from an image acquired after administering the reagent to remove regions appearing in both images, such as autofluorescence, thus obtaining an image in which only detected fluorescence affected by administration of the reagent is extracted. Because tumors have many (leaky) blood vessels, regions having many areas of high intensity in the extracted image can be recognized as tumors.

### BACKGROUND OF THE INVENTION

1. Field of the Invention

The present invention relates to an examination method for examining biological tissue in vivo.

This application is based on Japanese Patent Application No. 2007-105936, the content of which is incorporated herein by reference.

2. Description of Related Art

Techniques for in vivo examination of the internal state of a biological specimen, such as a small animal, from outside the body by using light are important in medical research and other fields. In particular, it is important to determine the position and size of a tumor by image analysis.

A known example of this type of examination method is fluorescence observation using green fluorescent protein (GFP) or a similar material. Recently, methods for observing near-infrared fluorescence, which has good transmittance characteristics in a body, have also become available, for example, the examination method disclosed in the Publication of Japanese Patent No. 3896176.

In this examination method, fluorescence is produced by forming a composite of a near-infrared fluorescent dye having low toxicity but substantially no fluorescence in an aqueous solution, such as indocyanine green, and a suitable high-density lipoprotein or the like. Based on this approach, external fluorescence imaging is performed by introducing the composite into a living organism to function as a near-infrared fluorescent tracer, irradiating the organism with excitation light, and detecting the near-infrared fluorescence from the tracer.

At near-infrared wavelengths, there is also autofluorescence from the skin, internal organs, and so on of the mouse, and it is likely that this autofluorescence will also be detected with the related art method. The effects of this autofluorescence cannot be ignored during examination.

In addition, with the related art method, some solution may remain in the body for a long time after administration, though it depends on the concentration of the solution and the amount administered. Therefore, subsequent administration is not possible until after a certain period of time has passed, and hence, this method may not be suitable for quantitative examination.

### BRIEF SUMMARY OF THE INVENTION

The present invention has been conceived in light of the problems described above, and an object thereof is to provide a method for quantitative examination of biological tissue using a near-infrared fluorescent dye, while eliminating the effects of autofluorescence.

In order to achieve the object described above, the present invention provides the following solutions.

A first aspect of the present invention is an in vivo examination method using at least a near-infrared fluorescent dye, comprising a step of acquiring an examination image before administering the dye; a step of administering the dye; a step of acquiring an examination image after administering the dye; and a step of performing image processing using the images acquired before and after administering the dye.

With the first aspect of the present invention, it is possible to perform quantitative examination of biological tissue using a near-infrared fluorescent dye.

A second aspect of the present invention is an in vivo examination method using at least a near-infrared fluorescent dye, comprising a step of administering the dye; a step of acquiring an examination image immediately after administering the dye; a step of acquiring an examination image a predetermined duration after administering the dye; and a step of performing image processing using the images acquired immediately after administering the dye and after the predetermined duration.

With the second aspect of the present invention, it is possible to perform quantitative examination of biological tissue with a near-infrared fluorescent dye.

A third aspect of the present invention is an in vivo examination method wherein a solution formed of at least a near-infrared fluorescent dye and a detection-object targeting agent is used.

With the third aspect of the present invention, it is possible to perform quantitative examination of biological tissue, even with a solution composed of a near-infrared fluorescent dye and a detection-object targeting agent.

A fourth aspect of the present invention is an in vivo examination method wherein the detection-object targeting agent is an antibody.

With the fourth aspect of the present invention, it is possible to perform quantitative examination of biological tissue even with a solution composed of a near-infrared fluorescent dye and an antibody.

A fifth aspect of the present invention is an in vivo examination method wherein a solution formed of at least a near-infrared fluorescent dye and an unbound molecular compound is used.

With the fifth aspect of the present invention, it is possible to perform quantitative examination of biological tissue even with a solution composed of a near-infrared fluorescent dye and an unbound molecular compound.

A sixth aspect of the present invention is an in vivo examination method wherein the unbound molecular compound is a polysaccharide.

With the sixth aspect of the present invention, it is possible to perform quantitative examination of biological tissue even with a solution composed of a near-infrared fluorescent dye and a polysaccharide.

A seventh aspect of the present invention is an in vivo examination method wherein the unbound molecular compound is a polypeptide.

With the seventh aspect of the present invention, it is possible to perform quantitative examination of biological tissue even with a solution composed of a near-infrared fluorescent dye and a polypeptide.

With the present invention, it is possible to provide a fluorescence observation method using a near-infrared fluorescent reagent, while eliminating the effects of autofluorescence.

### BRIEF DESCRIPTION OF THE SEVERAL VIEWS OF THE DRAWINGS

FIG. 1 is an example apparatus configuration (on-axis illumination) according to an Example of the present invention.

FIG. 2 is an example apparatus configuration (oblique illumination) according to an Example of the present invention.

FIG. 3 is a flowchart of a single examination sequence in Example 1 of the present invention.

FIG. 4 is a diagram for explaining the Example of the present invention.

FIG. 5 is a flowchart of a single examination sequence in Example 2 of the present invention.

### DETAILED DESCRIPTION OF THE INVENTION

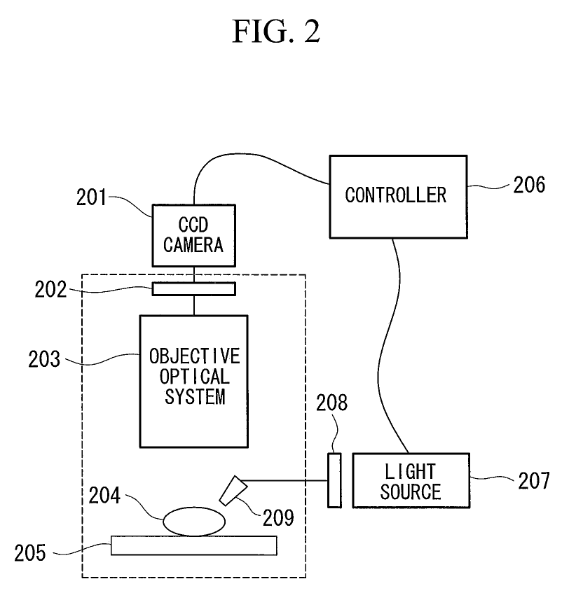

A first embodiment of the present invention will be described below using the following Example with reference to FIGS. 2 to 4.

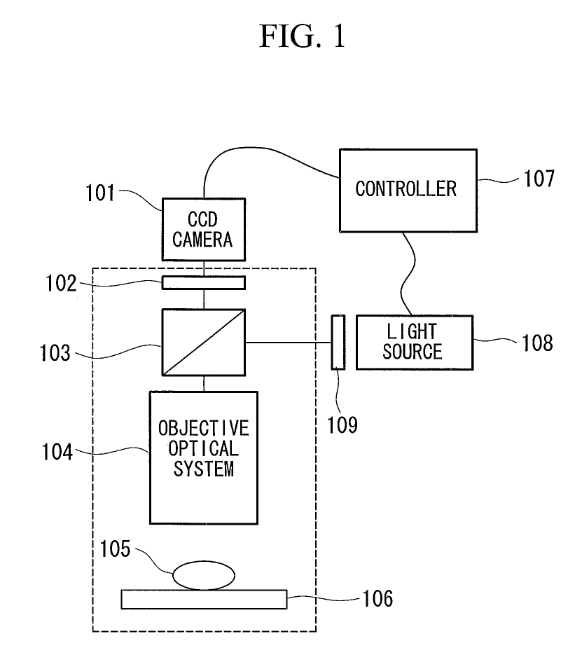

In this Example, by way of illustration, a near-infrared fluorescent reagent is administered to a live small laboratory animal, near-infrared fluorescence is converted to an image, and the area of a tumor is measured. An example configuration of the examination apparatus used in the present invention is shown in outline in FIG. 1 or FIG. 2. FIG. 1 shows an on-axis illumination setup, and FIG. 2 shows an oblique illumination setup.

First, FIG. 1 will be described. Of light emitted from a light source 108, only light of wavelengths that excite the near-infrared fluorescent dye are transmitted through a filter 109, reflected at a dichroic mirror 103, pass through an objective optical system 104, and irradiate a specimen 105 on a stage 106. Fluorescence generated at the specimen 105 passes in reverse through the objective optical system 104 and is transmitted through the dichroic mirror 103. Unwanted light components thereof are cut at a filter 102, and the remaining fluorescence is detected by a CCD camera 101.

A controller 107, which is a computer such as a standard PC, controls the image-capture conditions for the CCD camera 101, image conversion and display of the captured image, the intensity of the light source 108, and so on. It can also perform image processing and image calculation. If a plurality of the filters 102, filters 109, and dichroic mirrors 103 are provided and can be electrically switched, the controller 107 also controls that function. If the objective optical system 104 is provided with a zoom function or if the stage 106 is a motorized type, the controller 107 also controls those functions.

Next, FIG. 2 will be described. Of light emitted from a light source 207, only light of wavelengths that excite the near-infrared fluorescent dye are transmitted through a filter 208, pass through a fiber 209, and irradiate a specimen 204 on a stage 205. Fluorescence generated in the specimen passes through the objective optical system 203. Unwanted light components thereof are cut at a filter 202, and the remaining fluorescence is detected by a CCD camera 201. The light detected by the CCD camera 201 is transferred to a controller 206 where it is converted to an image. The controller 206 has the same functions as the controller 107 in FIG. 1.

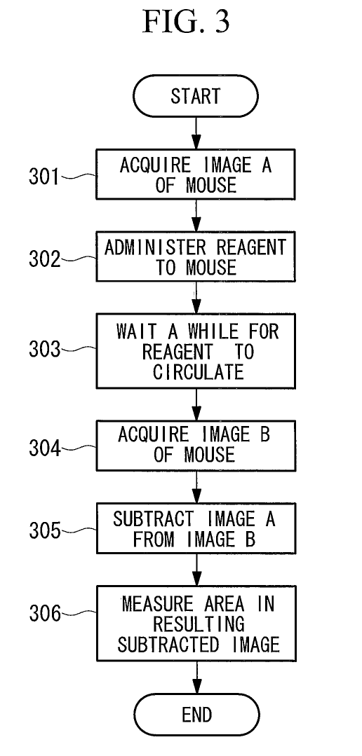

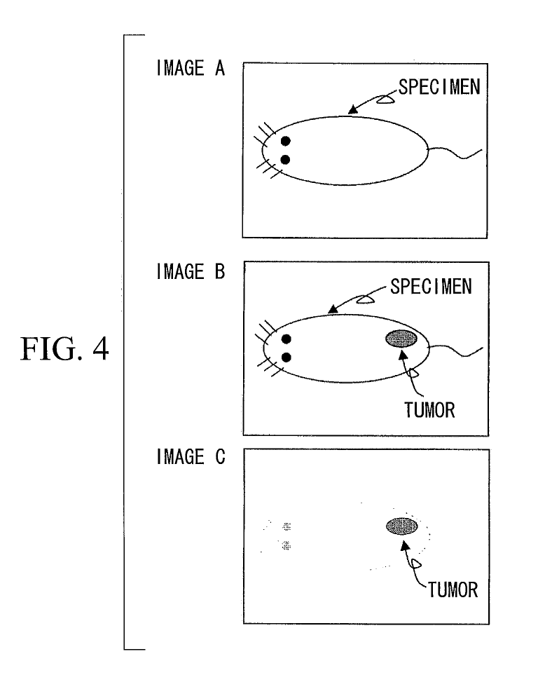

The examination procedure is as follows. The subject to be examined is a mouse implanted subcutaneously with tumor cells. Growth of the tumor is observed over time by measuring the area of the tumor. A single examination procedure is described here using FIGS. 3 and 4.

For example, Alexa Fluor (registered trademark) 680 dextran, 10,000 MW (Invitrogen) is used as the near-infrared fluorescent reagent. This reagent is composed of an infrared fluorescent dye and a polysaccharide.

The mouse is anesthetized with, for example, a gas anesthetic using isoflurane (not shown). An image of the mouse containing a portion where a tumor is present is initially acquired while no near-infrared fluorescent reagent is administered (Step 301 in FIG. 3). This acquires an image of the mouse alone, for example, image A shown in FIG. 4. When acquiring the image, the excitation filter, the detection filter, the dichroic mirror, and so forth are configured as required in order to observe near-infrared fluorescence.

Once the image has been acquired, the near-infrared fluorescent agent is administered to the mouse (Step 302 in FIG. 3). After a brief period of time, the reagent is circulated in the mouse body and tumor. The reagent signal of entire tumor vasculature, tumor vascular leak, etc., can be detected to a level allowing the external shape of the tumor to be recognized (Step 303 in FIG. 3). In this state, an image of the mouse in the same position is acquired again under the same image-acquisition conditions as used before administering the reagent (Step 304 in FIG. 3). Both the mouse and the tumor are observed in the image, for example, the image B shown in FIG. 4. Thus, images of the mouse before and after administering the reagent are acquired.

Next, the area of the tumor is measured. The image A before administering the reagent is subtracted from the image B after administering the reagent (Step 305 in FIG. 3). By doing so, parts that appear in both images, such as autofluorescence etc., are removed, and it is possible to obtain an image in which only the detected fluorescence affected by administering the reagent is extracted, as shown in image C in FIG. 4, for example. Because a tumor contains many (leaky) blood vessels, an area in the extracted image having many high-intensity regions is recognized as a tumor. Therefore, it is possible to measure the area of the tumor with a standard area measuring method, for example, a method for measuring the area by using a threshold value specified in advance and counting the number of pixels above the threshold value (Step 306 in FIG. 3).

After examination, once the effects of the anesthetic have worn off, the mouse wakes up and returns to normal activity.

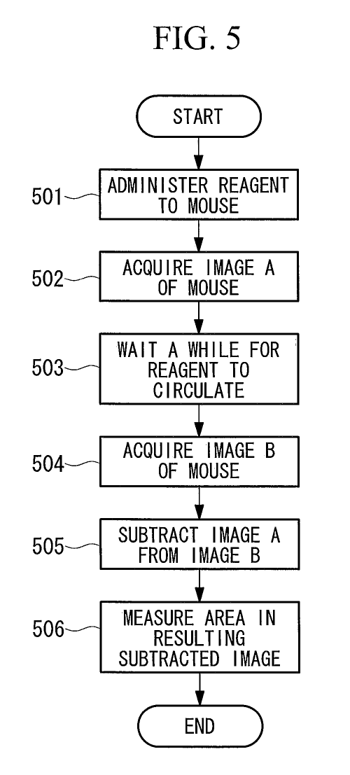

Next, a second embodiment will be described using the following Example with reference to FIG. 5.

The examination apparatus and subject to be examined are the same as in Example 1. However, the procedure for a single examination is different; therefore, that aspect will be described with reference to FIG. 5.

First, the mouse is anesthetized with a gas anesthetic using isoflurane, for example (not shown). Once anesthetized, a near-infrared fluorescent reagent is administered to the mouse (Step 501 in FIG. 5). Immediately after administering the near-infrared fluorescent reagent, an image of the mouse containing a portion where a tumor is present is initially acquired (Step 502 in FIG. 5), that is, an image of the mouse alone, as shown in image A in FIG. 4 for example. When acquiring the image, the excitation filter, detection filter, and dichroic mirror are configured as required, similarly to Example 1, in order to observe the near-infrared fluorescence.

After a brief period of time, the reagent is circulated in the mouse body, and fluorescence from the entire tumor can be detected to a level allowing the external shape of the tumor to be recognized (step 503 in FIG. 5) by the reagent signal of tumor microvasculature, tumor vascular leak, etc. In this state, an image of the mouse in the same position is acquired again under the same image-acquisition conditions as used immediately after administering the reagent (Step 504 in FIG. 5). Both the mouse and the tumor are observed in the image, for example, the image B shown in FIG. 4. Thus images of the mouse both immediately after administering the reagent and after a predetermined duration are acquired.

Next, the area of the tumor is measured. The image A acquired immediately after administering the reagent is subtracted from the image B acquired a predetermined duration after administering the reagent (Step 505 in FIG. 5). By doing so, parts that appear in both images, such as autofluorescence etc., are removed, and it is possible to obtain an image in which only the detected fluorescence affected by administering the reagent is extracted, as shown in image C in FIG. 4, for example. Because a tumor contains many (leaky) blood vessels, an area in the extracted image having many high-intensity regions can be recognized as a tumor. Therefore, it is possible to measure the area of the tumor with a standard area measuring method (Step 506 in FIG. 5).

After examination, once the effects of the anesthetic have worn off, the mouse wakes up and returns to normal activity.

The image-acquisition conditions used when acquiring the images before and after administering the reagent in the first embodiment and the images both immediately after administering the reagent and after a predetermined duration in the second embodiment are the same. However, if the images are acquired under different image-acquisition conditions, the signal intensity and contrast may be adjusted to equalize the one of the mouse tissue etc. at the same positions in the images before performing image calculations.

In the first embodiment, subtraction is performed using the image acquired before and after administering the reagent, and in the second embodiment, subtraction is performed using the images acquired immediately after administering the reagent and after a predetermined duration; however, the image calculation is not limited thereto. Also, although the area of a tumor is calculated, the subject to be examined is not restricted to just tumors; blood vessels, bones, and so on may also be examined. The parameter to be calculated is not limited to the area; the perimeter length, circularity, Feret's diameter and so on may also be analyzed.

Furthermore, in the first embodiment and the second embodiment, only two images are acquired; however, because images before administering the reagent and after administering the reagent may be acquired, moving images or time-lapse images may be acquired from before administration until a predetermined duration after administration, and some of those images may be used for the measurement.

In the first embodiment and the second embodiment, two-dimensional images are acquired; however, three-dimensional images may be acquired and their volumes calculated.

What is claimed is:

1. An in viva examination method using at least a near-infrared fluorescent dye, comprising:
a step of acquiring an examination image before administering the dye;
a step of administering the dye;
a step of acquiring an examination image after administering the dye; and
a step of performing image processing using the images acquired before and after administering the dye.

2. An in viva examination method using at least a near-infrared fluorescent dye, comprising:
a step of administering the dye;
a step of acquiring an examination image immediately after administering the dye;
a step of acquiring an examination image a predetermined duration after administering the dye; and
a step of performing image processing using the images acquired immediately after administering the dye and after the predetermined duration.

3. An in viva examination method according to claim 1, wherein a solution composed of at least a near-infrared fluorescent dye and a detection-object targeting agent is used.

4. An in viva examination method according to claim 3, wherein the detection-object targeting agent is an antibody.

5. An in viva examination method according to claim 1, wherein a solution composed of at least a near-infrared fluorescent dye and an unbound molecular compound is used.

6. An in viva examination method according to claim 5, wherein the unbound molecular compound is a polysaccharide.

7. An in viva examination method according to claim 5, wherein the unbound molecular compound is a polypeptide.

8. An in viva examination method according to claim 2, wherein a solution composed of at least a near-infrared fluorescent dye and a detection-object targeting agent is used.

9. An in viva examination method according to claim 8, wherein the detection-object targeting agent is an antibody.

10. An in viva examination method according to claim 2, wherein the detection-object targeting agent is an antibody.

11. An in viva examination method according to claim 10, wherein the unbound molecular compound is a polysaccharide.

12. An in viva examination method according to claim 10, wherein the unbound molecular compound is a polypeptide.
